(19) United States
(12) Patent Application Publication
(10) Pub. No.: US 2012/0022613 A1
(43) Pub. Date: Jan. 26, 2012

(54) Transcutaneous Modulated Power Link for a Medical Implant

(75) Inventor: Werner Meskens, Mechelen (BE)

(73) Assignee: Cochlear Limited, Macquarie University (AU)

(21) Appl. No.: 13/257,168

(22) PCT Filed: Mar. 15, 2010
(86) PCT No.: PCT/AU10/00300

§ 371 Date: Oct. 10, 2011

(30) Foreign Application Priority Data
Mar. 16, 2009 (AU) 2009901122

(51) Int. Cl. A61F 11/04

(52) U.S. Cl. 607/57

(57) ABSTRACT

A medical implant system such a Direct Acoustic Cochlear Stimulation and method for generating a transcutanous link between an external module and an internal module. A signal is generated in the external module by modulating an input signal using pulse modulation and then further modulating the pulse modulated signal using digital modulation. In the internal module, the received signal is processed using digital demodulation, the digitally demodulated signal being applied to the input of an amplifier to generate a control signal to control an actuator of the implant. A power component may also be extracted from the received signal in the internal module.

### PRIORITY

The present application claims priority from Australian Provisional Patent Application No. 2009901122 filed on 16 Mar. 2009.

The entire content of this document is hereby incorporated by reference.

### INCORPORATION BY REFERENCE

The following documents are referred to in the present description:
U.S. Pat. No. 6,240,318 entitled “Transcutaneous Energy Transmission System With Full Wave Class E Rectifier”
International Patent Application No. PCT/AU2005/001801 (WO2006/058368) entitled “Implantable Actuator For Hearing Aid Applications”

The entire content of each of these documents is hereby incorporated by reference.

### FIELD

The present invention relates to a modulated power link for use with an implanted mechanical actuator.

### BACKGROUND

A variety of medical implants exists to assist users who suffer from a loss of one or more senses, such as sight or hearing.

Users who suffer from a loss of hearing may be assisted by various devices. One such device is a hearing aid, which amplifies and/or clarifies surrounding sounds and directs this into the user's ear. Another device is a cochlear implant, which provides stimulating electrical energy directly to the user's auditory nerves in the cochlea. Another type of hearing device which may be used if the user's cochlea is functioning well, but the middle ear is defective, is a mechanical actuator which provides direct mechanical vibrations to a part of the user's hearing system such as the middle ear, inner ear, or bone surrounding the hearing system. One example of such a device is known as a Direct Acoustic Cochlear Stimulation (DACS) system, in which the actuator operates directly on the cochlea.

A DACS system consists of an external part which receives and processes surrounding acoustic energy, to control an internal part, including the actuator. The external part receives and processes the acoustic energy into data and converts this into signals that can be transmitted wirelessly through the skin of the user via a coil transmitter in the external part. The internal, implanted part has a receive coil for receiving the transmitted data and converting this into control signals that control the movement of the actuator, which acts directly onto a part of the user's hearing system such as a part of the inner ear (e.g. the stapes) or directly onto the oval window of the cochlea. This then generates vibrations on the cochlea fluid which stimulates hair cells which then stimulates nerves connected directly to the brain to be perceived as sound, as is the normal function of the cochlea.

As will be appreciated, the internal part requires power to operate. In some types of DACS systems, the power is provided by a local power supply, however, in some systems, the power may be provided via a transcutaneous power link transmitted and received through the external and internal coils respectively.

### SUMMARY

In one aspect, a method is disclosed of processing an external signal for transmitting transcutaneously to an internal module of an implanted medical device. The method comprises receiving the external signal, converting the external signal into an electrical signal, modulating the electrical signal using pulse modulation to provide a pulse modulated signal, and then modulating the pulse modulated signal using a digital modulation to provide a transmission signal for transmitting to the internal module.

In another aspect, a method is disclosed of providing a control signal to an actuator of an implanted medical device. The method comprises receiving a transmission signal comprising a pulse modulated electrical signal further modulated using digital modulation, digitally demodulating the received transmission signal to remove the digital modulation and then applying the demodulated signal to an input of an amplifier to provide the control signal for the actuator.

In a further aspect, an external module of a medical implant system is disclosed for processing an external signal for transmitting transcutaneously to an implanted internal module of the medical implant system. The external module comprises an input for receiving the external signal and for converting the received external signal into an electrical signal, a pulse modulator for modulating the electrical signal to provide a pulse modulated signal, a digital modulator for modulating the pulse modulated signal to provide a transmission signal and then an antenna for transmitting the transmission signal.

In yet a further aspect, an internal module for a medical implant system is disclosed. The internal module comprises a receiver for receiving a transmission signal comprising a pulse modulated electrical signal further modulated using digital modulation, a digital demodulator for digitally demodulating the received transmission signal and an amplifier for receiving the digitally demodulated signal and for providing a control signal to an actuator.

In still yet a further aspect, a Direct Acoustic Cochlear Stimulator system is disclosed. The Direct Acoustic Cochlear Stimulator comprises an external module comprising an input for receiving an external signal and for converting the received external signal into an electrical signal, a pulse modulator for modulating the electrical signal to provide a pulse modulated signal, a digital modulator for modulating the pulse modulated signal to provide a transmission signal and an antenna for transmitting the transmission signal. The system also comprises an internal module comprising a receiver for receiving the transmission signal, a digital demodulator for digitally demodulating the received transmission signal and an amplifier for receiving the digitally demodulated signal and for providing a control signal to an actuator.

### DRAWINGS

FIG. 1 - shows one example of a Direct Acoustic Cochlear Stimulator system to which the various aspects of the present invention may be applied;

FIG. 2 - shows a broad method of processing an external signal in medical implant system;

FIG. 3 - shows one example implementation of the method of FIG. 2 in an external module of the medical implant system;

FIG. 4 - shows a broad method of providing a control signal to an actuator of a medical implant;

FIG. 5 - shows one example implementation of the method of FIG. 4 in an internal module of the medical implant system;

FIG. 6 - shows a broad arrangement of a medical implant system incorporating the external module of FIG. 3 and the internal module of FIG. 5;

FIG. 7 - shows a high-level system block diagram of the components of an example stimulation system;

FIG. 8 - shows a more detailed system block diagram of an example arrangement of a stimulation system;

FIG. 9 - shows a time diagram of PWM using a triangular wave and FSK generation;

FIG. 10 - shows an alternative embodiment of the arrangement of FIG. 8;

FIG. 11 - shows an example of a PWM modulation circuitry;

FIG. 12 - shows an example of the various aspects of the present invention applied to a T-BAHA system;

FIG. 13 - shows an example of some components in the internal module of a DACS system according to one aspect of the present invention;

FIG. 14 - shows a circuit diagram of an internal part with FSK demodulation by a phase-locked loop (PLL);

FIG. 15 - shows a circuit diagram of an internal part with FSK demodulation using slope detection;

FIG. 16 - shows a circuit diagram of an internal part with FSK demodulation using slope detection; a

FIG. 17 - shows an alternative embodiment to the arrangement of FIG. 16;

FIG. 18 - shows a block diagram of ne specific embodiment of a medical implant system;

FIG. 19 - shows an example circuit arrangement of the PWM of FIG. 18;

FIG. 20 - shows an example circuit arrangement of the OOK modulator of FIG. 18;

FIG. 21 - shows the input and outputs waveforms of the circuit of FIG. 20;

FIG. 22 - shows an example circuit arrangement of the coil driver of FIG. 18; and

FIG. 23 - shows an example circuit arrangement of the internal module of FIG. 18.

### DETAILED DESCRIPTION

While the various aspects will largely be described with reference to a DACS system, it will be understood that the various aspects may be applied to any direct electromechanical stimulation implant system including any middle ear, inner ear and transcutaneous bone anchored hearing aid implant systems.

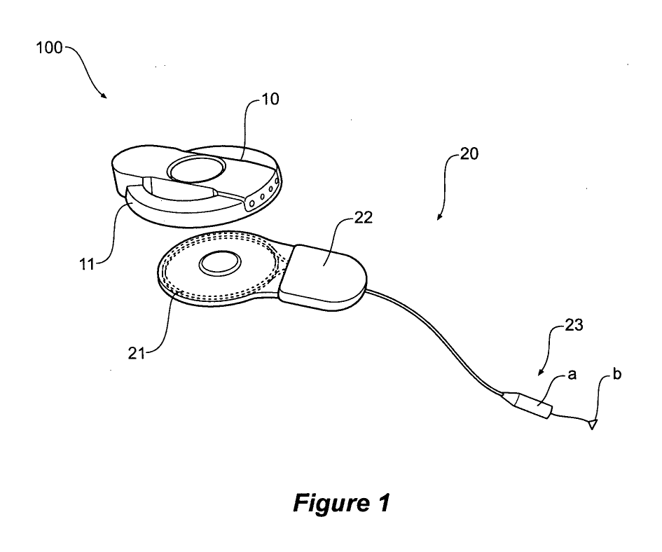

FIG. 1 shows an example of a DACS system 100 to which the various aspects described herein may be applied. The system 100 includes an external part 10 and an internal part 20. External part 10 includes a microphone (not shown) or other transducer for detecting and receiving acoustic energy from the environment, a processing system (not shown) inside the casing of the external part, and a wireless antenna such as a coil 11 for transmitting the processed signals wirelessly. The internal part 20, to be implanted in the user, includes an internal antenna or receiving coil 21, receiver and processor electronic circuits embedded in an implant body 22, and an actuator 23. In use, the processed signals transmitted by external antenna or coil 11 are received by the internal antenna or coil 21 and to provide control signals for actuator 23 to vibrate to generate the acoustic stimulation as required. The end 23b of the actuator 23 may be connected to any suitable part of the user's hearing system such as the stapes, or may in fact be connected directly to the oval window of the cochlea by way of a prosthetic stapes.

The outer material of the implant body 22 is biocompatible and provides a strong protective housing for the internal electronics. If the material has non-magnetic or has low electrical conductive properties on the operating FSK carrier frequency, then the internal antenna or coil 21 could even be integrated inside the implant body.

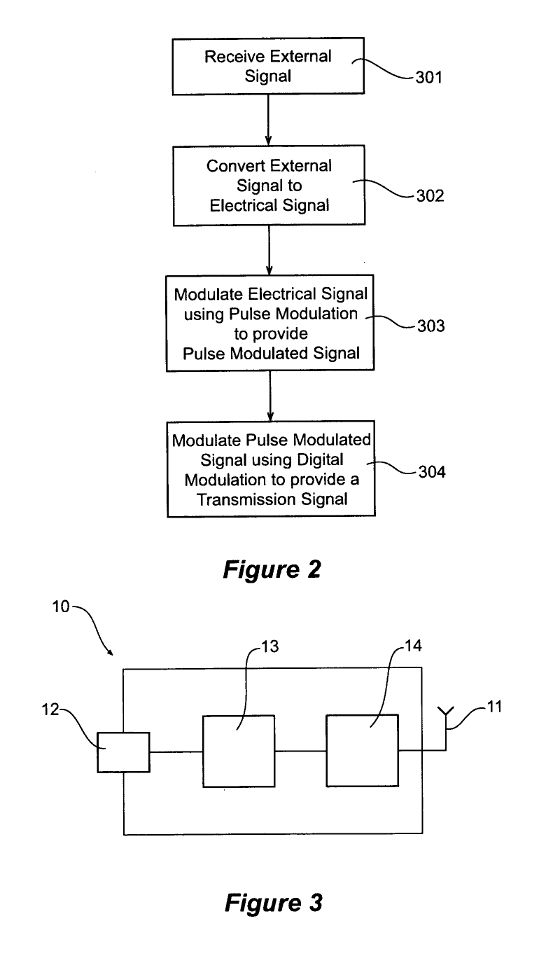

FIG. 2 shows a broad method of processing an external signal (such as an acoustic signal or a test signal) for use in a medical implant system such as a DACS system, according to one aspect. At step 301, the external signal is received. At step 302, the received external signal is converted into an electrical signal. At step 303, the electrical signal is modulated using pulse modulation to provide a pulse modulated signal. As will be understood by the person skilled in the art, pulse modulation involves the modulation of pulses of a pulse signal with a modulating signal (such as the electrical signal). Various forms of pulse modulation may be used, including but not limited to, Pulse Amplitude Modulation (PAM), Pulse Width Modulation (PWM), Pulse position Modulation (PPM), Pulse Code Modulation (PCM), Differential PCM (DPCM), Adaptive DPCM (ADPCM), Delta Modulation, Sigma-Delta Modulation and Pulse Density Modulation (PDM).

At step 304, the pulse modulated signal is then modulated using digital modulation to provide a transmission signal which may then, in one example, be transmitted to an internal module of the medical implant system. Again as will be understood by the person skilled in the art, various forms of digital modulation may be used, including but not limited to, Frequency Shift Keying (FSK), Amplitude Shift Keying (ASK), On-Off Keying (OOK), Phase Shift Keying (PSK), Quadrature Amplitude Modulation (QAM), Minimum Shift Keying (MSK), Continuous Phase Modulation (CPM), and Pulse Position Modulation (PPM).

FIG. 3 shows a broad arrangement for one implementation of the method described above by external module 10 of a medical implant system such as a DACS system. Shown there is external module 10, comprising an input such as an acoustic transducer such as a microphone 12, for receiving an acoustic signal such as sound from the surrounding environment. Alternatively, or in combination, input 12 may be a test button or other user interface that the user or an operator may use to generate a test or other signal. In the case where the input is an acoustic transducer, the transducer 12 converts the acoustic signal into an electrical signal. Connected to the transducer 12 is a first modulator, in this case a pulse modulator 13, which in use, receives as an input, the electrical signal from transducer 12. Pulse modulator 13 modulates the received electrical signal to provide a pulse modulated signal. As described above, various types of pulse modulators may be used, including but not limited to, Pulse Amplitude Modulator (PAM), Pulse Width Modulator (PWM), Pulse Position Modulator (PPM), Pulse Code Modulator (PCM), Differential PCM (DPCM), Adaptive DPCM (ADPCM), Delta Modulator, Sigma-Delta Modulator and Pulse Density Modulator (PDM).

Following the pulse modulator 13 is a digital modulator 14, which modulates the pulse modulated signal provided by pulse modulator 13. Digital modulator 14 modulates the pulse modulated signal to provide a transmission signal for subsequent transmission to the internal module 20 via antenna 11 as will be described in more detail below. Again as will be understood by the person skilled in the art, various types of digital modulators may be used, including but not limited to, Frequency Shift Keying (FSK) modulator, Amplitude Shift Keying (ASK) modulator, On-Off Keying (OOK) modulator, Phase Shift Keying (PSK) modulators, Quadrature Amplitude Modulation (QAM) modulator, Minimum Shift Keying (MSK) modulator, Continuous Phase Modulation (CPM) modulator, and Pulse Position Modulation (PPM) modulator.

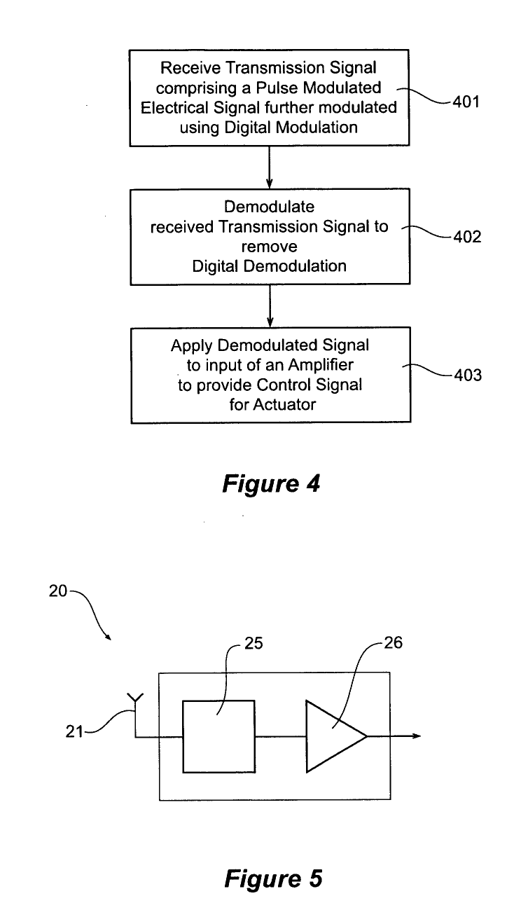

FIG. 4 shows a broad method of providing a control signal to an actuator of an implanted medical device. In step 401, a transmission signal is received. The transmission signal comprises a pulse modulated electrical signal which is further modulated using digital modulation. Such a transmission signal may be generated by the method and apparatus as described above with reference to FIGS. 2 and 3 for example. In step 402, the received transmission signal is digitally demodulated to remove the digital demodulation. The type of digital demodulation used would be appropriate to the type of digital modulation used, and may be any suitable including those described above.

In step 403, the digitally demodulated signal is then applied to the input of an amplifier to then provide the control signal for use with an actuator of for example, a DACS system.

FIG. 5 shows one possible implementation of an internal module for carrying out the method described above. Shown in FIG. 5 is internal module 20 comprising a receiver 21, such as an internal antenna or internal coil for receiving a transmission signal, a digital demodulator 25 for digitally demodulating the transmission signal to provide the digitally demodulated signal, and an amplifier 26, an input of which is connected to the output of digital demodulator 25. The output of amplifier 26 provides the control signal which may be applied to an actuator.

In one form, the digital demodulator 25 is an FSK demodulator. In one form, the amplifier 26 is a Class D amplifier. In one form, the internal module 20 also comprises the actuator 23.

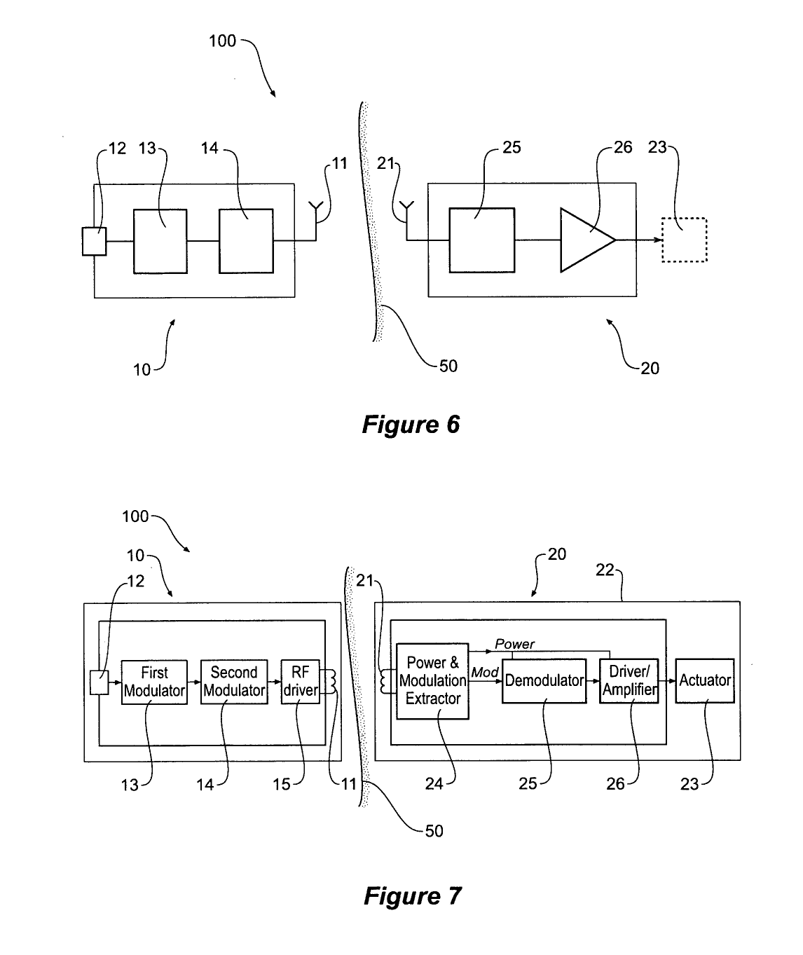

FIG. 6 shows broad arrangement of a medical implant system such as a DACS system 100 incorporating the external module 10 of FIG. 3 and the internal module 20 of FIG. 5 described above. In this example, there is shown external module 10 with input 12 for receiving an external signal, pulse modulator 13 for pulse modulating an electrical signal provided by input 12, digital modulator 14 for digitally modulating the pulse modulated signal from pulse modulator 13 to provide the transmission signal and external antenna 11 for transmitting the transmission signal transcutaneously through tissue 50 of the user.

Shown implanted within the user, is internal module 20. Internal module 20 comprises internal antenna 21 for receiving the transmission signal transmitted transcutaneously by external antenna 11, digital demodulator 25 for removing the digital demodulation from the transmission signal, and amplifier 26, for receiving the digitally demodulated signal from digital demodulator 25 and providing the control signal for use in controlling the actuator. FIG. 6 shows that in one example. The internal module 20 may also comprise actuator 23, but in other example or embodiments, actuator 23 may be provided separately and later connected to internal module 20.

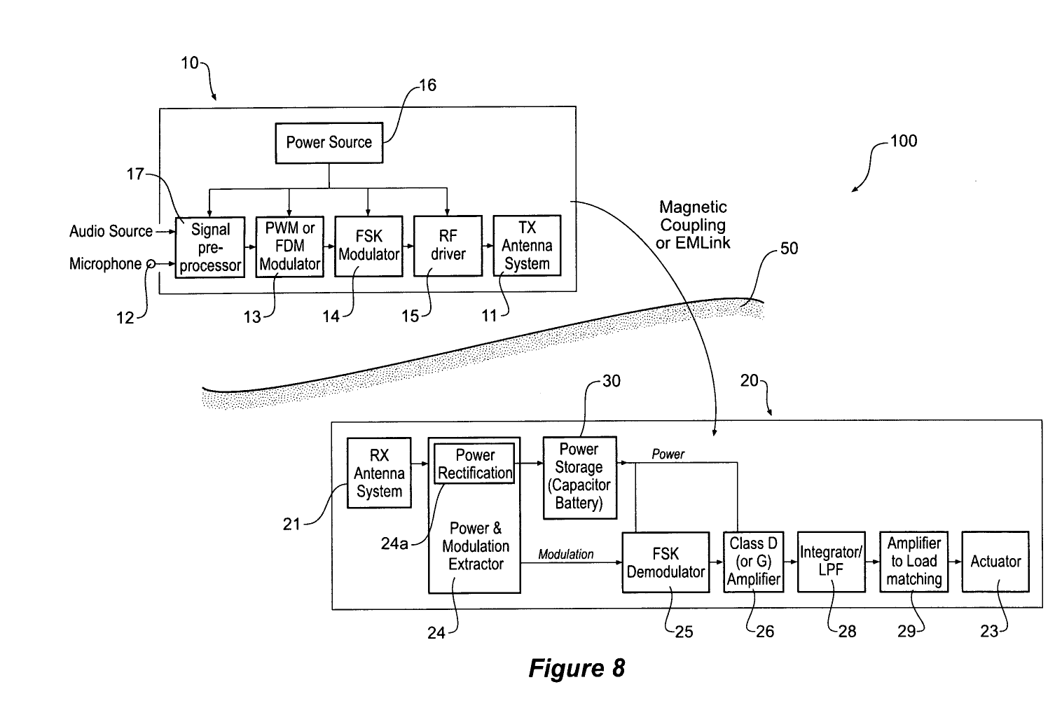

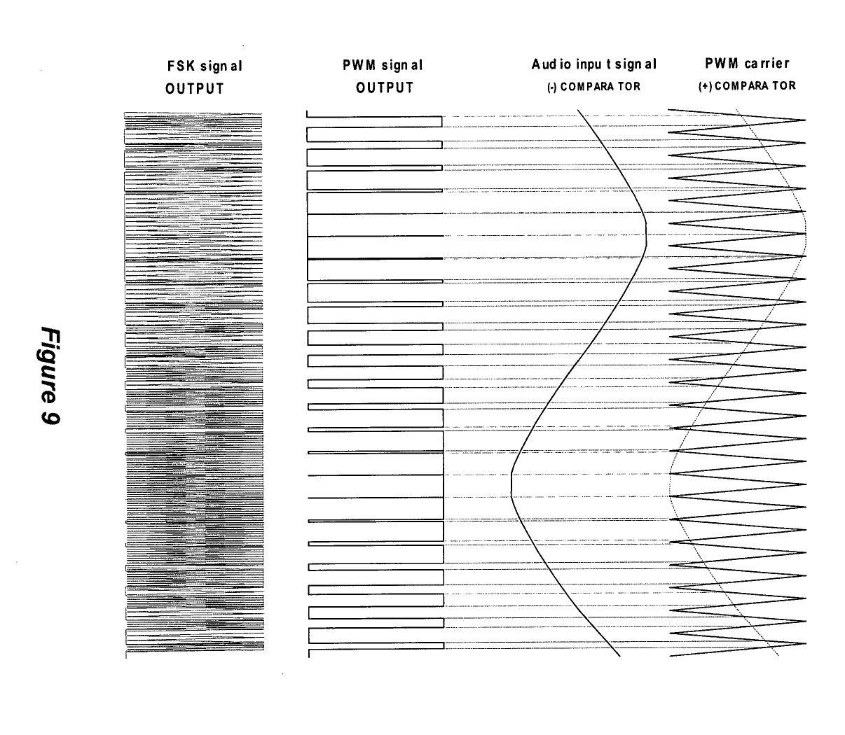

FIG. 7 shows a simplified system block diagram of a medical implant system, a transcutaneous electromechanical stimulator system 100 such as a DACS system or a T-BAHA system, showing practical implementations and modifications of the external module 10 of FIG. 3 and internal module 20 of FIG. 4 and the system of FIG. 6 described above. The two modules are separated by a layer of tissue 50 such as a portion of the user's scalp.

External module 10 includes an acoustic transducer 12 such as a microphone. The microphone 12 receives acoustic energy from the surrounding environment and translates this energy into electrical signals. These electrical signals are then input to a first modulator 13, in this case, a pulse modulator, to provide a pulse-modulated signal. The pulse-modulated signal is then applied to the input of a second modulator 14, in this case a digital modulator 14 to provide a further modulated signal or transmission signal. The transmission signal is then amplified in RF driver 15 and then applied to an antenna or coil 11 for wireless transmission through the layer of tissue 50 to the internal, implanted module 20. The internal module 10 may also include an audio pre-processing block (not shown) improving or optimizing the audio signal quality prior to modulation.

The internal module 20 includes internal antenna 21 as a receiving antenna or coil, for receiving the transmission signal from the transmitting antenna or coil 11. In this embodiment, the received signal is then applied to a power and modulation extracting block 24, which extracts power from the received transmission signal for powering a demodulator 25 and driver/amplifier 26 of the internal module 20. An additional post-processing circuitry (not shown) may also be included in the implant body 22. The demodulated and amplified signal is applied to the actuator 23 referred to above.

In one embodiment, the pulse modulator 13 is a Pulse Width Modulation (PWM) modulator and the digital modulator 14 is a Frequency Shift Keying (FSK) modulator. In another embodiment, the pulse modulator 13 is a Pulse Density Modulation (PDM) Modulator.

FIG. 8 shows a more detailed block diagram of a transcutaneous modulated power link including components in the internal module 20 for demodulating and processing the data components in the transmitted transmission signal as well as extracting the power component in the transmitted transmission signal.

In FIG. 8, external module 10 includes acoustic transducer or microphone 12, audio or signal pre-processing block 17 for conditioning the electrical signals generated by microphone 12, and for applying the conditioned signal to Pulse Width Modulation (PWM) or Pulse Density Modulation (PDM) modulator 13, which generates a PWM or PDM signal. This signal is then applied to the input of a Frequency Shift Keying (FSK) modulator 14 to generate a further modulated signal as the transmission signal which is then applied to external antenna or coil 11 for wireless transmission through tissue 50 to the internal module 20.

Internal module 20 includes the internal or receive antenna or coil 21 which receives the wirelessly transmitted transmission signal from the external module 10 and produces an electrical signal for use by the internal module 20. This electrical signal is applied to the input of rectification system 24a, which extracts a power component from the electrical signal which may be used to provide power to one or more of the remaining components of the internal module 20. In one form, the extracted power signal may be stored on a power storage device 30 such as a capacitor or small battery.

Any suitable extraction circuit may be used as will be understood by the person skilled in the art. This includes a simple rectification circuit with one or more diodes. An example of another suitable power rectification circuit is shown in U.S. Pat. No. 6,240,318, previously incorporated by reference.

The received electrical signal in this example, is also processed to extract the control information or control signal to actuate the mechanical actuator 23. In this example, the received electrical signal is applied to the input of a Frequency Shift Keying (FSK) demodulator 25 which removes the FSK modulation applied in the external module 10. This FSK demodulated signal is then applied directly to a class D amplifier 26. The output of this is then applied to a low pass filter or integrator 28, the output of which is adapted or optimized to the impedance of the actuator by the amplifier 26 to load matching block 29. Depending on the type of actuator load the matching block 29 and low-pass filter 28 could be created by a single block with combined functionality e.g. a passive network of inductors and/or capacitors. The output of this is then applied to the mechanical actuator 23 which generates stimulating vibrations in accordance with the signals applied. A suitable mechanical actuator is described in International Patent Application No. PCT/AU2005/001801 (WO2006/058368) previously incorporated by reference.

In one example, the transcutaneous link emanating from the external device or module 10 is an FSK modulated signal that is derived from the PWM or PDM signal. Therefore the auditory signal emanating from a microphone or any other audio source is first transformed into a PWM or PDM signal and then connected to the input of the FSK modulator. The modulated FSK signal is placed on an antenna or coil powering the implant with a quasi-continuous envelope signal enabling a maximized power transfer from an external power source to the implant.

In one embodiment, the implant module contains a Class D amplifier 26 that can directly be driven by the FSK demodulator. This provides a significant simplification on the electrical circuit of the implant or internal module 20. Class D amplifiers have a theoretical efficiency of 100%. The output of the Class D amplifier 26 is connected to the actuator 23 via a suitable filter network (for example, RC or LC low pass filter, integrator) to block the PWM carrier and recover the original audio signal.

A further advantage of the arrangement of FIG. 8 is that the FSK demodulator 25 of the implant device or internal module 20 is less susceptible to signal distortion caused by sinking the necessary implant power from the secondary or internal antenna or coil 21 (part of the implant resonance tank). Thus the transcutaneous FSK signal containing power and PWM or PDM signal is very robust against clipping which can be caused by non-linear elements and power rectification of the implant.

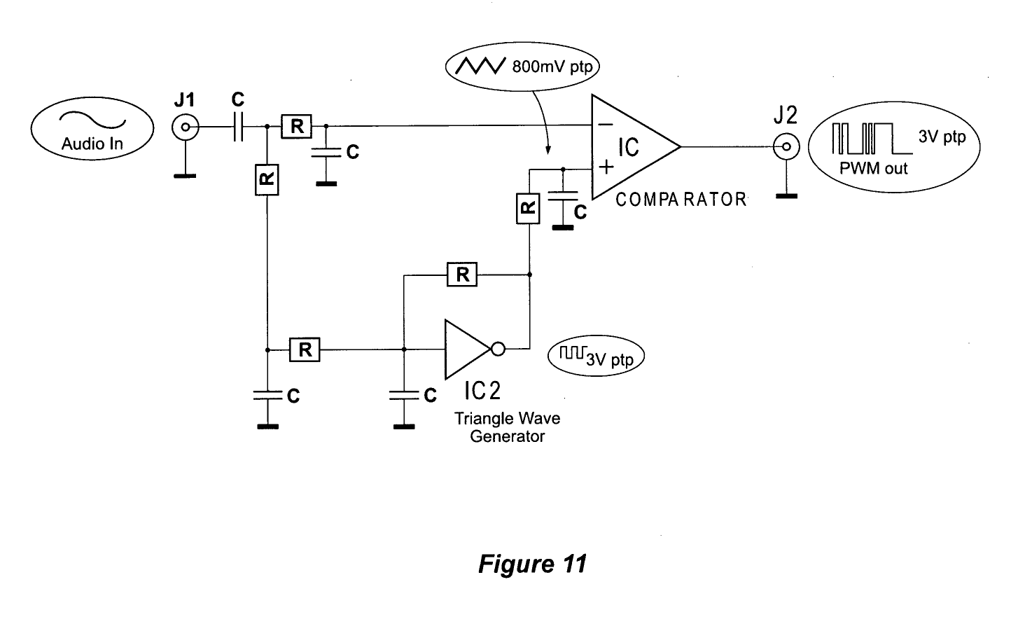

PWM compares the analogue audio input signal to a triangular or sawtooth shaped waveform at a fixed carrier frequency well above the audio range (e.g. 200 KHz). The comparator's output gives a stream of pulses with variable width. The width of each pulse is proportional to the amplitude of the audio input signal. FIG. 9 illustrates the creation of PWM and one embodiment of a PWM modulation circuitry is depicted in FIG. 11.

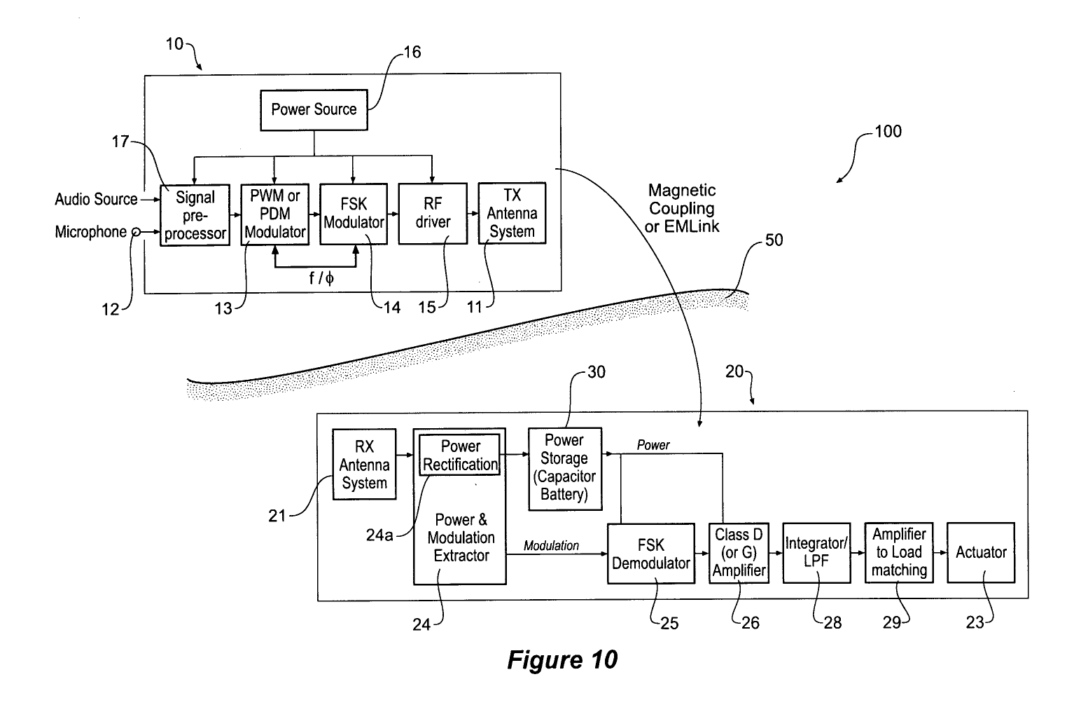

FIG. 10 shows a modification of the arrangement of FIG. 8. In this embodiment, as in the embodiment of FIG. 8, external module 10 includes acoustic transducer or microphone 12, audio or signal pre-processing block 17 for conditioning the electrical signals generated by microphone 12, and for applying the conditioned signal to Pulse Width Modulation (PWM) or Pulse Density Modulation (PDM) modulator 13, which generates a PWM or PDM signal. This signal is then applied to the input of a Frequency Shift Keying (FSK) modulator 14 to generate a further modulated signal or transmission signal which is then applied to antenna or coil 11 for wireless transmission through tissue 50 to the internal module 20.

In this modification, a fixed relationship or frequency ratio between the frequencies used by the PWM modulator and FSK modulator is provided as seen in FIG. 10. This provides an improved signal to noise ratio of the demodulated signal in the internal module. This fixed relationship is in one form, equal to an integer ratio between the FSK carrier and PWM frequency. This can be accomplished by a divider or a phase locked loop (PLL) with phase comparator.

PDM is accomplished by a sigma-delta modulator giving a variable rate of pulses which is proportional to the amplitude of the audio input signal in a given time window. Each pulse has the same time duration.

In other examples, the FSK modulation may be replaced by other forms of modulation such as Continuous Phase Frequency Shift Keying (CPFSK), Phase Shift Keying (PSK), Amplitude Phase Shift Keying (ASK) or On Off Keying (OOK). The Class D amplifier 26 may still be driven directly by an OOK demodulator. Using an OOK modulation would still reduce the number of electrical components on the demodulator block of the implant in comparison with prior art arrangements. A simple OOK envelope detector could be made using a diode loaded to an RC parallel circuit.

In other examples, the Class D amplifier may be replaced by a Class G amplifier.

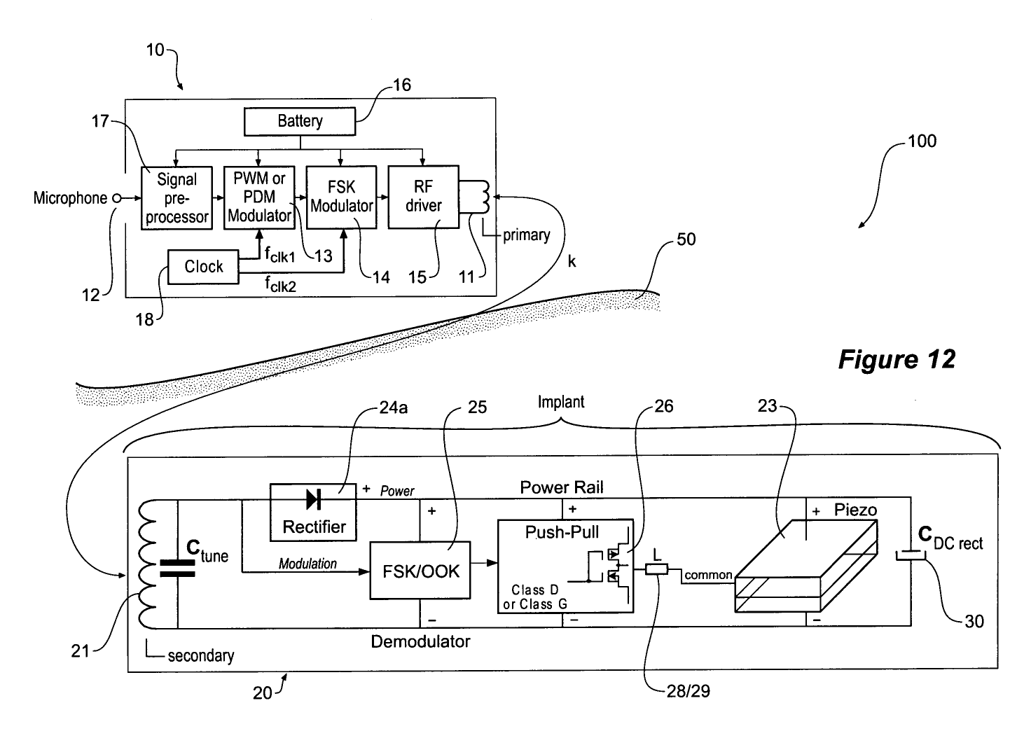

FIG. 12 shows an application of the various aspects of the present invention to a Transcutaneous Bone Anchored Hearing Aid (T-BAHA) system 100. Shown in FIG. 12 is external module or device 10 including microphone 12, audio filter and amplifier (signal conditioning) 17, PWM modulator 13, FSK modulator 14, radio frequency (RF) driver 15 and antenna 11.

The internal device or module 20 includes receive antenna 21, power rectifier circuit 24a, FSK demodulator 25 which is driving a Class D or Class G amplifier 26, which drives actuator 23. In this example, actuator 23 is a piezoelectric device.

FIG. 12 shows a simplified form of the implant 20 containing an implant coil, a power rectification block (synchronous or diode half-wave/full wave rectification), an FSK demodulator, a Class D audio amplifier and an actuator. In this example, all components may be embedded in one or more biocompatible implant casings. There could also optionally be an implantable power source (e.g. battery) to be charged over the transcutaneous link here described. In this example the frequency sources or clocks used by the modulators of the external module 10 are derived from a common frequency source or clock unit 18.

In one embodiment, there is a fixed relationship between the frequencies generated by the PWM modulator and FSK modulator improving the signal to noise ratio of the demodulated signal in the internal module. The clock unit (18) could also contain frequency dividers, multipliers and PLLs, guaranteeing the fixed relationship between f_{clk1} and f_{clk2}. The circuit complexity remains in the external module, with a simplified arrangement of the internal module. When using an OOK modulator there could be used an OOK carrier frequency with a constant frequency relationship to the frequency source used to establish the PWM signal. In one embodiment, the frequency ratio between the frequency sources of the digital modulator and the pulse modulator is an integer (e.g.. 200 KHz PWM and 5 MHz OOK carrier).

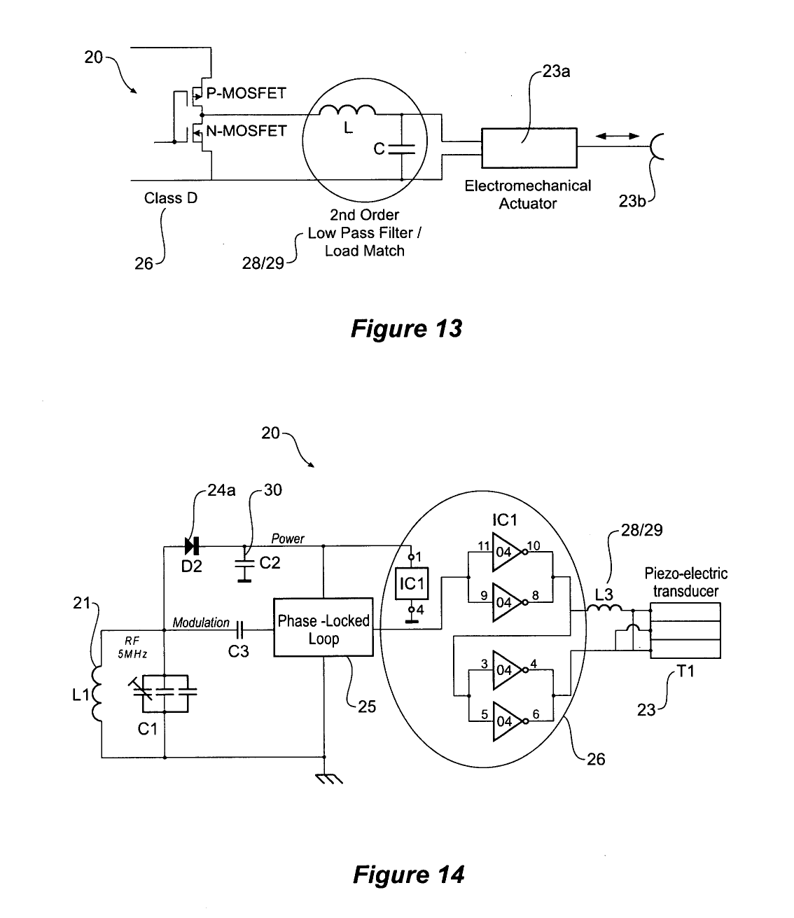

FIG. 13 shows a particular arrangement of another embodiment of back-end components in the internal module or device 20 of a DACS system. Shown here is Class D amplifier 26 with an LC low pass filter arrangement on its output, driving actuator 23. In this example, the end 23b of actuator 23 is connected to the stapes of the user's middle ear.

FIGS. 14 to 17 show different electrical schematic examples of the internal part or internal module 20.

FIG. 14 shows a circuit diagram of an internal module 20 with FSK demodulation by a phase-locked loop (PLL) 25. PLL detectors are well known to the person skilled in the art.

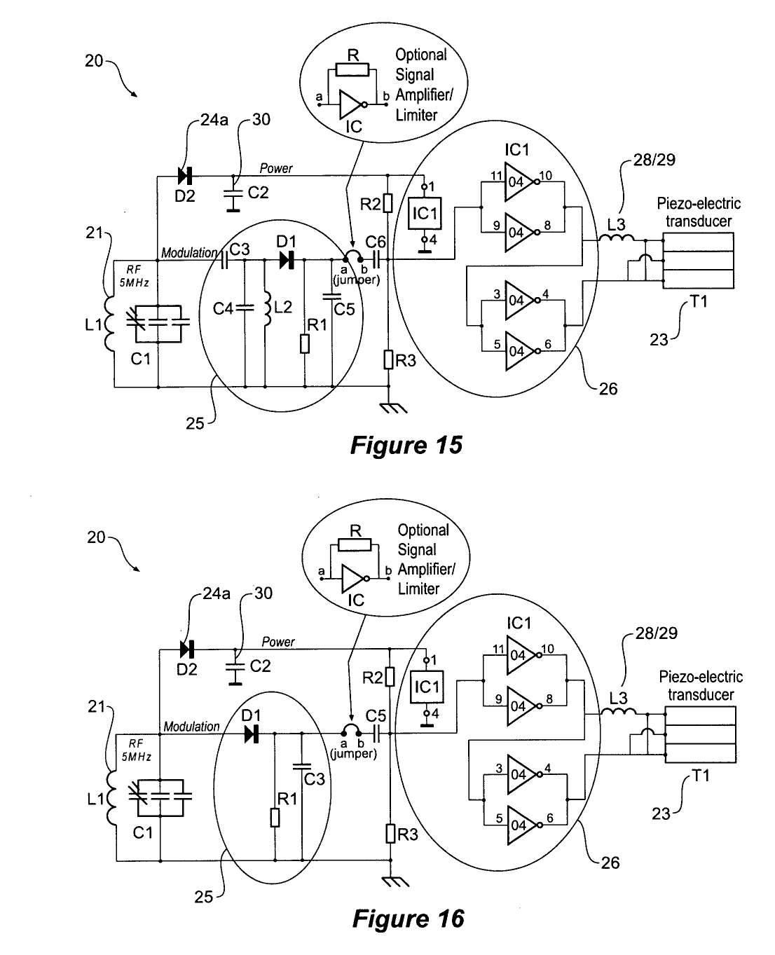

FIG. 15 shows a circuit diagram of an internal module 20 with FSK demodulation by slope detection. The LC tank (L2 and C4) is above or below the nominal FSK carrier RF frequency. Frequency variation on one sloping side of the LC tuning curve gives amplitude variation. This signal can be directly connected to a class D amplifier 26.

FIG. 16 shows a circuit diagram of an internal module 20 with OOK demodulation using an envelope diode detector using a simple RC network (R1 and C3).

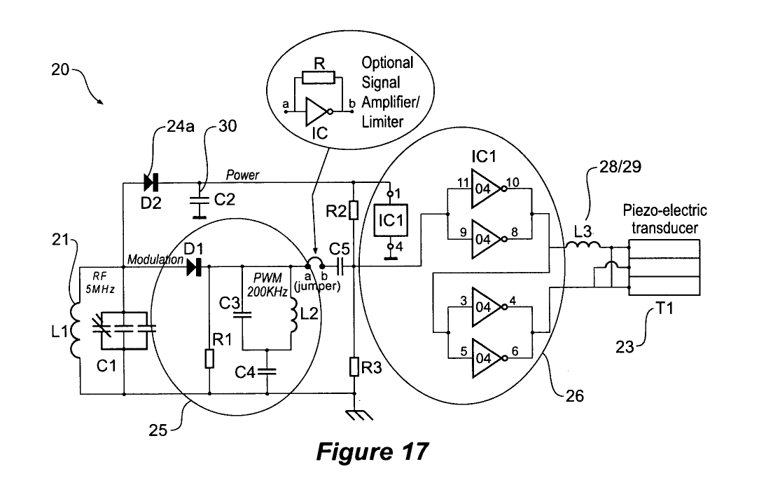

FIG. 17 shows a circuit diagram of an internal part with OOK demodulation using an envelope diode detector optimized for 200 KHz PWM carrier (L2, C3 resonance at 200 KHz). A simple RC network may also be used. The OOK demodulated signal is connected directly to the Class-D amplifier 26. This amplifier consists of push-pull MOSFETs in half- or full bridge architecture. For the practical implementation of the Class-D amplifiers logic inverters may be used.

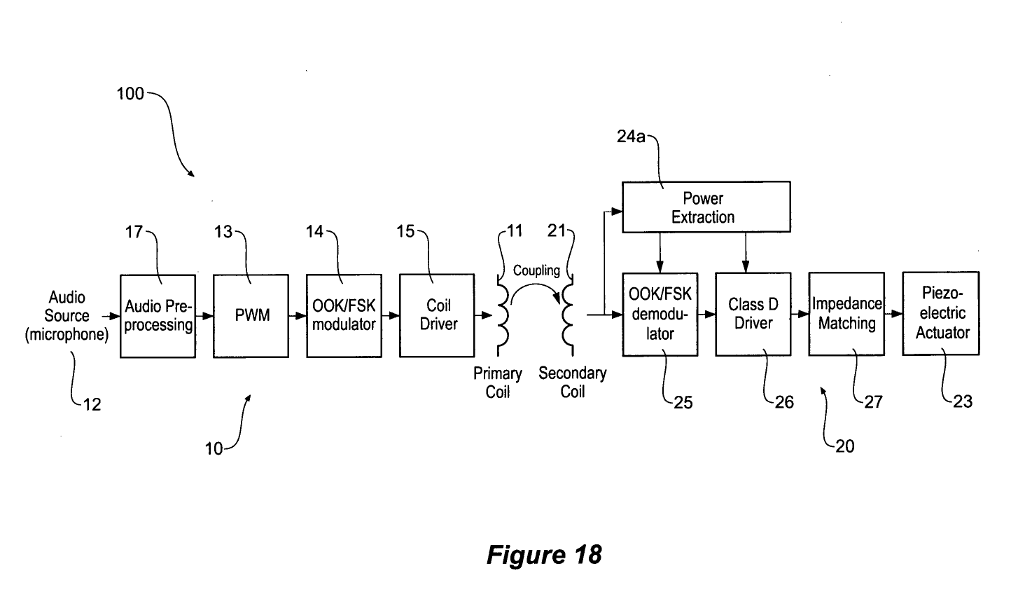

FIGS. 18 to 23 show a specific implementation of one embodiment using OOK modulation and demodulation. A 200 kHz PWM scheme is used, over a 5 MHz RF link. FIG. 18 shows a block diagram of the major components of medical device system 100 with external module 10 and internal module 20. In this example, external module 10 comprises input or microphone 12, an audio pre-processing block 17, pulse modulator (PWM) block 13, digital modulator as an OOK modulator 14, a coil driver block 15 and external antenna or primary coil 11. Any suitable audio pre-processor 17 may be used and the workings of which are well known to the person skilled in the art and will not be described in any further detail. The external module 10 may be powered by an external battery providing for example, about 24 mW, with about 3V operating voltage and about 8 mA DC current.

Internal module 20 in this example, comprises internal antenna or secondary coil 21, power extraction block 24a, digital or OOK demodulator 25, Class D amplifier 26, impedance matching block 27 and piezo-electric actuator 23.

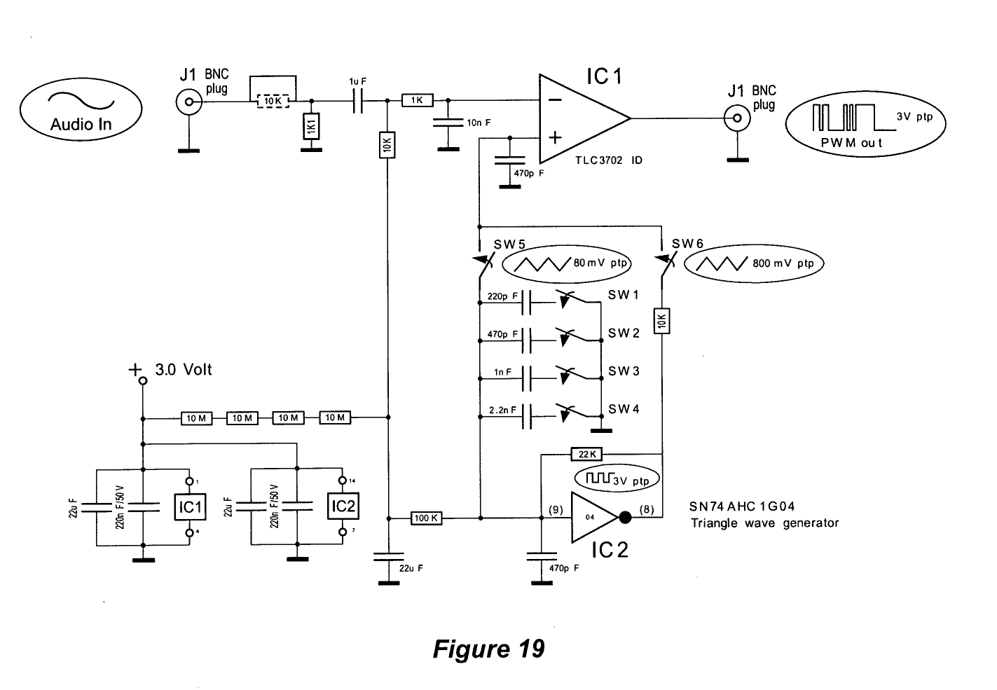

FIG. 19 shows pulse modulator (PWM) block 13 in detail. The PWM is provided by a logic gate inverter (IC2) together with the capacitor network and switches SW1 to SW6 as shown. Switches SW5 and SW6 are normally closed. The coarse PWM frequency is set by selection of SW1 to SW4. The generated output is a triangular wave of about 800 mV peak to peak. This triangular wave is provided as an input to comparator IC 1 (for example TLC3 7021D). Applied to the inverting input of IC1 is the processed audio signal. The output of comparator IC1 is a PWM wave of about 3V peak to peak. Any ripple or noise on the 3V power supply is filtered out by placement of 22uF and 220nF capacitors on Vdd (voltage supply pin) of IC1 and IC2.

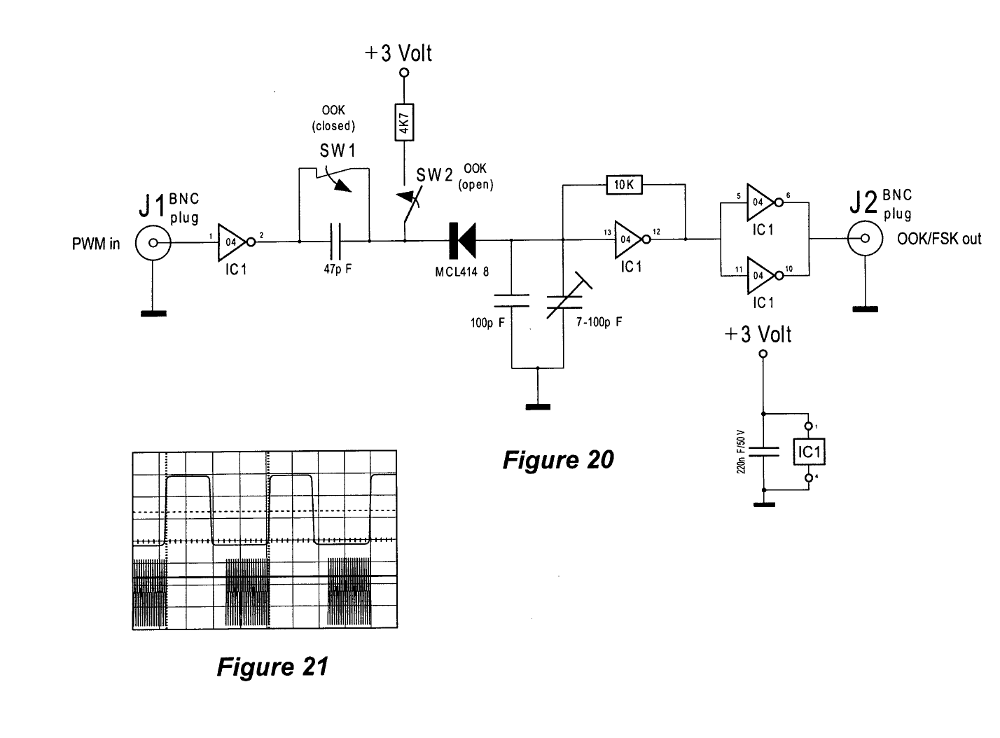

FIG. 20 shows an example circuit for digital modulator 14. This circuit uses two inverter logic gates (74AC04) to generate an OOK or FSK signal modulated signal at 4.5 to 6.5 MHz frequency, The output is buffered by two additional inverters placed in parallel. The RC oscillator of the modulator is formed by a resistor R=10K, two capacitors C=100 pF in parallel with 7-100 pF and a single inverter (pin 13/12). In the case of an OOK modulator SW1 is closed and SW2 opened. When the diode MCL4148 is not conducting or reversely polarized the oscillator will start-up and becomes operational. This happens when zero volt is applied at the input of J1 (pin 2 of IC1 is +3 Volts and pin 13 is about +1.5V). In this case 3V is applied at the input of J1 the diode starts to conduct on the RC oscillator will stop.

The input and output of the OOK modulator 14 are shown in FIG. 21. The upper trace shows the input signal at J1 and the lower trace shows the output signal at J2.

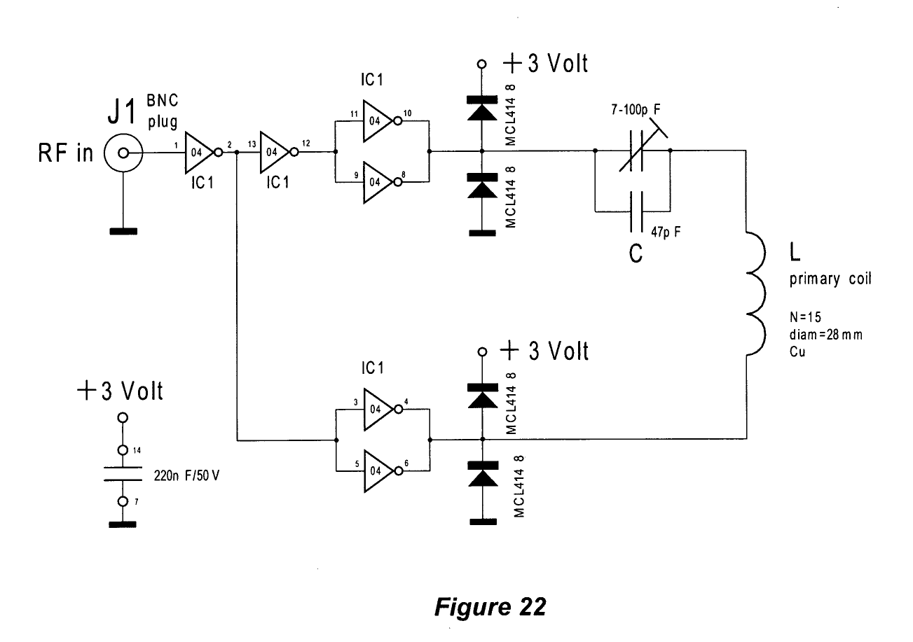

FIG. 22 shows an example circuit for the coil driver 15 with a differential output The primary coil L is tuned to about 5 MHz resonance by a series capacitor C (47 pF in parallel with 7-100 pF). The inverter gates of the differential output drivers are placed 2 by 2 in parallel to provide sufficient current going through the series resonant circuit LC. The coil driver 15 in this example uses a total of 6 inverter logic gates (74AC04).The 4 MCL4148 diodes protect the circuit from high transients caused by the LC tank or ESD.

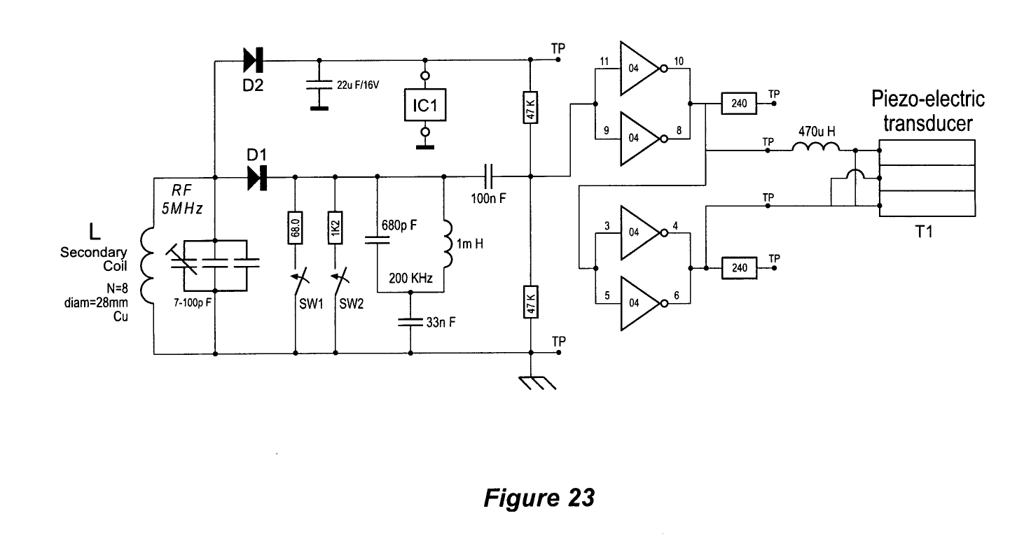

Turning now to the details of the internal module or implant 20, FIG. 23 shows a basic example circuit for the implant. The received OOK transmission signal transmitted by external antenna or primary coil 11 is received by internal antenna or secondary coil 21, which in this example comprises an 8-turn copper coil of about 28 mm diameter. Diode D2 provides the rectification to extract the power component from the transmission signal to provide power to the remainder of the implant circuit. The transmission signal is applied to the input of PWM demodulator provided by D1, switches SW1 and SW2 with respective resistors 680 or /and 1K2 and the resonance tank tuned to about 200 kHz. D1 and D2 are e.g.. standard Si-diodes such as MCL4148.

The output of this block is applied to the Class D amplifier 26 provided by the 74AC04 logic inverters, with a 2 to 6 volt range. The Class D output is differential.

The additional 470 uH inductor is provided between the output of amplifier 26 and the piezo-electric transducer T1 providing the actuator 23 to limit the current at 250 kHz. Additional zener diodes or transorbs could be used to protect the circuit from over-voltages (not shown).

The various aspects of the present invention provide several advantages over current systems. For example, the arrangement shown allows much of the circuit complexity to remain in the external module 10, with a simplified arrangement of the internal module 20. The internal circuitry is simplified in one form by having the demodulator directly driving the amplifier. Furthermore, the arrangement does not require a separate PWM or PDM demodulator to remove the Pulse Width Modulation or Pulse Density Modulation of the original audio signal applied in the external module. The arrangements described herein may be used in a uni-directional system (i.e. power and data flow from the external module to the internal module) thus allowing for further simplification of the internal module.

The various aspects of the present invention have been described with reference to specific embodiments. It will be appreciated however, that various variations and modifications may be made within the broadest scope of the principles described herein.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

1. A method of processing an audio signal for transmitting transcutaneously to an internal module of an implanted medical device, the method comprising:
receiving the audio signal;
converting the audio signal into an electrical signal;
modulating the electrical signal using pulse modulation to provide a pulse modulated signal; and
modulating the pulse modulated signal using a digital modulation to provide a transmission signal for transmitting to the internal module.

2. A method as claimed in claim 1 wherein the pulse modulation is Pulse Width Modulation (PWM),

3. A method as claimed in claim 1 wherein the pulse modulation is Pulse Density Modulation (PDM).

4. A method as claimed in claim 1 wherein the digital modulation is Frequency Shift Keying (FSK).

5. A method as claimed in claim 1 wherein the digital modulation is Continuous Phase Frequency Shift Keying (CPFSK).

6. A method as claimed in claim 1 wherein the digital modulation is Phase Shift Keying (PSK).

7. A method as claimed in claim 1 wherein the digital modulation is On Off Keying (OOK).

8. (canceled)

8. A method as claimed in claim 1 wherein there is provided a frequency ratio between frequencies used in the pulse width modulation and the digital modulation.

9. A method as claimed in claim 8 wherein the frequency ratio is an integer.

10. A method of providing a control signal to an actuator of an implanted hearing device, the method comprising:
receiving a transmission signal comprising a pulse modulated electrical signal further modulated using digital modulation;
digitally demodulating the received transmission signal to remove the digital modulation; and
applying the demodulated signal to an input of an amplifier to provide the control signal for the actuator.

11. A method as claimed in claim 10 wherein the digital demodulation is FSK demodulation.

12. A method as claimed in claim 11 wherein the amplifier is a Class D amplifier.

13. A method as claimed in claim 12 further comprising extracting a power component from the received transmission signal.

14. An external module of a hearing implant system for processing an external signal for transmitting transcutaneously to an implanted internal module of the hearing implant system, the external module comprising:
an input for receiving the external signal and for converting the received external signal into an electrical signal;
a pulse modulator for modulating the electrical signal to provide a pulse modulated signal;
a digital modulator for modulating the pulse modulated signal to provide a transmission signal; and
an antenna for transmitting the transmission signal.

15. An external module as claimed in claim 14 wherein the pulse modulator is a Pulse Width Modulation (PWM) modulator.

16. An external module as claimed in claim 14 wherein the pulse modulator is a Pulse Density Modulation (PDM) modulator.

17. An external module as claimed in claim 14 wherein the digital modulator is a Frequency Shift Keying (FSK) modulator.

18. An external module as claimed in claim 14 wherein the digital modulator is a Continuous Phase Frequency Shift Keying (CPFSK) modulator.

19. An external module as claimed in claim 14 wherein the digital modulator is a Phase Shift Keying (PSK) modulator.

20. An external module as claimed in claim 14 wherein the digital modulator is an On Off Keying (OOK) modulator.

21. An external module as claimed in claim 14 wherein the external signal is an acoustic signal and the input is an acoustic transducer.

22. An external module as claimed in claim 14 wherein the pulse modulator and the digital modulator are related to each other by a fixed frequency ratio.

23. An external module as claimed in claim 22 wherein the fixed frequency ratio is an integer.

24. An internal module of a hearing implant system comprising:
a receiver for receiving a transmission signal comprising a pulse modulated electrical signal further modulated using digital modulation;
a digital demodulator for digitally demodulating the received transmission signal; and
an amplifier for receiving the digitally demodulated signal and for providing a control signal to an actuator.

25. An internal module as claimed in claim 24 wherein the digital demodulator is an FSK demodulator.

26. An internal module as claimed in claim 24 wherein the amplifier is a Class D amplifier.

27. An internal module as claimed in claim 24 further comprising the actuator.

28. An internal module as claimed in claim 24 further comprising a power extracting circuit for extracting a power component from the received transmission signal.

29. A Direct Acoustic Cochlear Stimulator system comprising:
an external module comprising:
an input for receiving an external signal and for converting the received external signal into an electrical signal;
a pulse modulator for modulating the electrical signal to provide a pulse modulated signal;
a digital modulator for modulating the pulse modulated signal to provide a transmission signal; and
an antenna for transmitting the transmission signal; and
an internal module comprising:
a receiver for receiving the transmission signal;
a digital demodulator for digitally demodulating the received transmission signal; and
an amplifier for receiving the digitally demodulated signal and for providing a control signal to an actuator.

30. A Direct Acoustic Cochlear Stimulator system as claimed in claim 29 wherein the digital demodulator is an FSK demodulator.

31. A Direct Acoustic Cochlear Stimulator system as claimed in claim 29 wherein the amplifier is a Class D amplifier.

32. A Direct Acoustic Cochlear Stimulator system as claimed in claim 29 further comprising the actuator.

33. A Direct Acoustic Cochlear Stimulator system as claimed in claim 29 further comprising a power extracting circuit for extracting a power component from the received transmission signal.
